(19) United States
(12) Patent Application Publication
(10) Pub. No.: US 2009/0291056 A1
(43) Pub. Date: Nov. 26, 2009

(54) Aqueous Based Cosmetic Compositions With Clear Or Translucent Non-Amidated Structuring Agent

(76) Inventors: John R. Castro, Huntington Station, NY (US); Arlene G. Ting-Jenulis, East Northport, NY (US); Katie Ann Frampton, Massapequa, NY (US)

Correspondence Address: THE ESTEE LAUDER COS, INC, 155 PINELAWN ROAD, STE 345 S, MELVILLE, NY, 11747, US

(21) Appl. No.: 12/123,820

(22) Filed: May 20, 2008

(51) Int. Cl. A61K 8/87; A61Q 1/10

(52) U.S. Cl. 424/70.7; 424/78.03

(57) ABSTRACT

Aqueous based compositions comprising at least one clear or translucent non-amidated structuring agent, at least one film forming polymer solubilized or dispersed in the aqueous phase, wherein the composition is substantially free of waxes.

### TECHNICAL FIELD

The invention is in the field of aqueous based cosmetic compositions for application to keratinous surfaces.

### BACKGROUND OF THE INVENTION

One common problem confronting cosmetics companies is the formulation of products that provide intense, vibrant color. Most color cosmetic compositions must contain some type of structuring agent, e.g. an ingredient that increases the viscosity of the composition to a thick liquid, semi-solid, or solid state. Most often these structuring agents are waxes. Most waxes are opaque and have the overall effect of matting the color cosmetic, which in turn tends to mute the vibrancy of the color. While certain types of clear structuring agents are known, they are not without their problems. Polyamides or silicone polyamides are an example of structuring agents that are clear or translucent. However, polyamides such as those sold by Arizona Chemical under the trade name Uniclear® are sometimes difficult to formulate into aqueous based compositions like emulsions because of their instability. Also, such polyamides and silicone polyamides are not always compatible with other commonly used cosmetic ingredients.

Accordingly, there is a need for aqueous based color cosmetic compositions in aqueous or emulsion form where the structuring agent used is clear or translucent, stable, and easily formulated into stable emulsions. It has been found that certain clear or translucent non-amidated gelling agents provide cosmetically acceptable aqueous based compositions, particularly when the compositions are substantially free of waxes, preferably free of waxes that are opaque.

It is an object of the invention to provide an aqueous based cosmetic composition that provides true and vibrant color.

It is a further object of the invention to provide aqueous based cosmetic compositions that contain non-amidated clear or translucent structuring agents and are substantially free of wax.

It is a further object of the invention to provide a method for making up keratinous surfaces by applying an aqueous based cosmetic composition substantially free of wax containing at least one non-amidated clear or translucent structuring agent.

It is a further object of the invention to provide a mascara composition and method for making up eyelashes wherein the composition comprises an aqueous based cosmetic composition substantially free of wax and containing at least one non-amidated clear or translucent structuring agent.

### SUMMARY OF THE INVENTION

The invention is directed to an aqueous based cosmetic composition comprising at least one non-amidated clear or translucent structuring agent wherein said composition is substantially free of wax.

The invention is further directed to an emulsion cosmetic composition comprising at least one non-amidated clear or translucent structuring agent wherein said composition is substantially free of wax.

The invention is further directed to a method for making up keratinous surfaces comprising applying to the surface an aqueous based composition comprising at least one non-amidated clear or translucent structuring agent wherein said composition is substantially free of wax.

The invention is further directed to a mascara composition comprising at least one non-amidated clear or translucent structuring agent wherein said composition is substantially free of wax.

### DETAILED DESCRIPTION

### I. Definitions

A. All percentages used herein are percentages by weight unless otherwise indicated.

B. “Clear” means, with respect to the structuring agent, the same as “transparent” and that the structuring agent permits both the passage of light and is sufficiently free of cloudiness or haziness to provide a clear view of what lies behind.

C. “Film Former” means an ingredient that, upon drying, will form a continuous or semi-continuous film on a keratinous surface, e.g. skin, hair, or nails.

D. “Non-amidated” means, with respect to the structuring agent, that it is substantially free of amine or amide moieties in its chemical structure, preferably completely free of amine or amide moieties, except in the case of natural materials of heterogeneous composition that may have trace amounts of amines or amides. Examples of amidated structuring agents specifically excluded include polyamides such as Uniclear™ or Sylvaclear™ sold by Arizona Chemical, or polyamides sold by Henkel under the Versamid™ trademark, as well as silicone polyamides sold by Dow Corning.

E. “Opaque” when used to describe a substance means that it does not permit the passage of light or a clear view of what lies behind.

F. “Substantially free of” means, with respect to the wax limitation, that the composition comprises less than about 5%, preferably less than about 3%, more preferably less than about 1%, most preferably 0% by weight of one or more waxes.

G. “Translucent” means, with respect to the structuring agent, that it permits passage of light but has sufficient cloudiness or haziness to prevent a clear view of what lies behind.

H. “Wax” or “Waxes” means, with respect to the “substantially free of” natural waxes having a melting point of about 50° C. or greater, such as carnauba, candelilla, ceresin, beeswax, ozokerite, bayberry, flower waxes, apple wax, jojoba wax, vegetable waxes, and the like.

### II. Aqueous Based Composition

The composition of the invention is in the form of an aqueous based composition such as a liquid, spray, gel, solution or suspension, or in the form of an emulsion such as an oil-in-water or water-in-oil emulsion. In the case where the composition is in the form of an aqueous gel, solution, or suspension, it may comprise from about 1 to 99.9%, preferably from about 5 to 98%, preferably from about 5 to 95% water in addition to other ingredients as further described herein. In the case where the composition is in the form of an emulsion, it may comprise from about 0.01 to 99%, preferably from about 0.05 to 95%, preferably from about 0.5 to 95% by weight of the total composition of water and from about 0.01 to 99%, preferably from about 0.05 to 95%, preferably from about 0.5 to 95% by weight of the total composition of oil.

### III. The Non-Amidated Structuring Agent

The invention comprises at least one non-amidated structuring agent, preferably present in an amount ranging from about 0.01 to 45%, preferably from about 0.05 to 40%, most preferably from about 0.1 to 35%. The structuring agent is preferably a solid or semi-solid at room temperature (25° C.), and may be a synthetic polymers, fatty acid soaps, natural rosin or rosin esters, and so on.

A. Polyolefins

Certain types of polyolefins are suitable non-amidated structuring agents. For example, polyolefins prepared by metallocene catalysis provide clear or translucent structuring agents. Particularly preferred are polyolefins sold by Clariant Corporation under the trademark Licocene™, in particular Licocene™ PP1302, PP1502 or PP1602 which are polyolefin copolymers having relatively low crystallinity which provide transparent structuring agents of a waxy consistency. These particular non-amidated clear or translucent structuring agents have the INCI name polypropylene. Another type of polyolefin that is also suitable has the INCI name polyethylene and is sold by Alzo International under the tradename AC Polyethylene 617A.

Also suitable in certain instances, e.g. when combined with other ingredients, are various types of polyalphaolefins that contain mixtures of linear alpha olefins with varying carbon chain lengths. Examples include C18-26 olefins, C20-24 olefins, C24-28 olefins, C30-45 olefins, and so on. One example is a mixture of ethylhexyl hydroxystearate/triethylhexyl trimellitate/C30-45 olefin, sold by Sensient Cosmetic Technologies under the trademark Clearwax™.

B. Homo- and Copolymers of Ethylenic Monomers

Also suitable as the non-amidated clear or translucent structuring agent are various homo- and copolymers of ethylenically unsaturated monomers such as those sold under the trademark Kraton™ by Kraton Polymers. Examples of such polymers include hydrogenated or non-hydrogenated homo- or copolymers of monomers such as styrene, butadiene, ethylene, propylene, isoprene, butylene, and the like, which copolymers may be in the form of block copolymers. Examples of such copolymers include hydrogenated styrene-isoprene copolymer, styrene-ethylene/butylene-styrene block copolymer, and the like. These block copolymers are often referred to by the following designations where the letter refers to the block polymer. Examples are SIBS (styrene-isoprene-butadiene-styrene), SEBS (styrene-ethylene-butadiene-styrene), SIS (styrene-isoprene-styrene), SBS (styrene-butadiene-styrene) and so on.

C. Hydroxy Substituted Fatty Acids

Also suitable as the non-amidated clear or translucent structuring agent are certain hydroxy-substituted fatty acids, for example, stearic acid substituted with one or more hydroxyl groups on any one or more of the carbon atoms. Particularly preferred is 12-hydroxystearic acid.

Also suitable are esters of such hydroxy acids, that is where the carboxylic acid is reacted with a straight or branched chain C₁₋₄₀ aliphatic or aromatic alcohol. Examples of such materials include ethyhexyl hydroxystearate, and the like.

D. Polysaccharides

Certain types of polysaccharides or derivatives thereof are also suitable as the clear or translucent structuring agent. In particular, certain plant based polysaccharides from the inulin family are preferred. Inulins may be derivatives by reacting one or more of the hydroxyl groups with C₁₋₄₀ straight or branched chain saturated or unsaturated carboxylic acids, preferably fatty carboxylic acids having from about 6 to 40 carbon atoms such as stearic, palmitic, myristic, oleic, linoleic, linolenic, behenic, acids and so on. Further examples of inulin or inulin derivatives include stearoyl inulin, sold by Tomen America under the trademark Rheopearl™. Also suitable are palmitoyl inulin or undecylenoyl inulin.

E. Rosin or Derivatives

Also suitable as the clear or translucent non-amidated structuring agent are by products of rosin feedstocks which may be derivatized by reacting with glycerin or other organic alcohols. One example is glyceryl rosinate or hydrogenated glyceryl rosinate made by Hercules Corporation and sold under the brand name Endere S Resin.

Particularly preferred are one or more clear or translucent structuring agents selected from polypropylene prepared by metallocene catalysis, rosin or glycerolate derivatives thereof, or mixtures thereof

### IV. Film Former

The composition comprises at least one film forming ingredient that is solubilized or dispersed in the aqueous phase. The film former may be present in amounts ranging from about 0.01 to 65%, preferably from about 0.1 to 60%, more preferably from about 0.5 to 50%. The film former may be in the form of a polymer. The film former may be hydrophilic or lipophilic. If the film former is dispersed in the aqueous phase it may be in the form of particles in aqueous dispersion. Suitable film formers include resinous plant extracts or synthetic or natural polymers. Additional film formers that are soluble or dispersible in the liphophilic phase of the emulsion may also be present and in the same general percentage ranges.

A. Resinous Plant Extracts

Examples of resinous plant extracts that provide film forming properties include shellac or derivative thereof.

B. Synthetic Polymers

1. Polyurethanes

A variety of synthetic polymers are suitable film formers including polyurethanes including those have the CTFA name Polyurethane 1, a copolymer of isophthalic acid, adipic acid, hexylene glycol, neopentyl glycol, dimethylolpropanoic acid, and isophorone diisocyanate monomers; Polyurethane 2, a copolymer of hexylene glycol, neopentyl glycol, adipic acid, saturated methylene diphenyldiisocyanate and dimethylolpropanoic acid monomers; Polyurethane 4, a copolymer of PPG-17, PPG-34, isophorone diisocyanate and dimethylolpropanoic acid monomers; Polyurethane 5, a copolymer of hexylene glycol, neopentyl glycol, adipic acid and isophorone diisocyanate monomers; Polyurethane 6, a copolymer of isophthalic acid, adipic acid, hexylene glycol, neopentyl glycol, dimethylolpropanoic acid, isophorone diisocyanate and bis-ethylaminoisobutyl-dimethicone monomers; Polyurethane 7, a copolymer of hexylene glycol, neopentyl glycol, adipic acid, isophorone diisocyanate and dimethylolpropanoic acid monomers; Polyurethane 8, which is a copolymer of polyethylene-poly(tetramethylene)glycol, propanoic anhydride, isophorone diisocyanate, and isophorone diamine; Polyurethane 9, a copolymer of adipic acid, toluene diisocyanate, propylene glycol, ethylene glycol and hydroxyethyl acrylate monomers; Polyurethane 10, a copolymer of isophorone diisocyanate, cyclohexanedimethanol, dimethylolbutanoic acid, polyalkylene glycol and N-methyl diethanolamine monomers; Polyurethane 11, a copolymer of Adipic Acid, 1,4-Butanediol, isophthalic acid, methylene bis-(4-cyclohexylisocyanate), neopentyl glycol and trimethylolpropane monomers; Polyurethane 12, a copolymer of adipic acid, 1,4-Butanediol, isophthalic acid, methylene bis-(4-cyclohexylisocyanate), neopentyl glycol, and trimethylolpropane monomers; Polyurethane 13, a copolymer of trimethylolpropane, dimethylol propionic acid, hexanediol, adipic acid, polyester diol, and isocyanato methylethylbenzene monomers; Polyurethane 14, a copolymer of isophorone diisocyanate, dimethylol propionic acid, and 4,4′-isopropylidenediphenol reacted with propylene oxide, ethylene oxide and PEG/PPG-17/3; Polyurethane 15, copolymer of isophorone diisocyanate, adipic acid, triethylene glycol, and dimethylolpropanoic acid; Polyurethane 16, a cross-linked condensation polymer formed from the addition polymerization of 2 moles of hexamethylene diisocyanate with 1 mole of polycaprolactonetriol terminated with 3 hydroxyl groups; Polyurethane 17, a complex polymer made by neutralizing hexylene glycol/neopentyl glycol/adipic/SMDI/DMPA copolymer with triethylamine in the presence of water; Polyurethane 18, a complex polymer formed by the reaction of m-tetramethylene diisocyanate, polybutylene glycol and dimethylol propionic acid; Polyurethane 19, a complex polymer formed by the reaction of m-tetramethylene diisocyanate, neopentyl glycol, trimethylol propane, dimethylol propionic acid and a polyester formed by condensing neopentyl glycol and adipic acid; and Polyurethanes 20, 21, 23, 24, 25, 26, 27, 28, or 32. Examples of such polyurethanes are sold by BASF under the trade name Luviset® such as Luviset P.U.R.®; or Noveon Consumer Specialties under the trademark Avalure®, such as Avalure® 405 or 425; or Kane International under the trademark KFilm®; or Wilde Cosmetics under the trademark Actilane®; or Cytec Industries under the trademark Cydrothane CG® and the like. Particularly preferred is Polyurethane 1, sold by BASF under the trademark Luviset P.U.R.®. this material is available as an aqueous emulsion in water having a solids content of 28 to 32% by weight of the polymer emulsion.

2. Acrylate or Methacrylate Homo- or Copolymers

Also suitable are various types of homo- or copolymers of monomers such as acrylic acid, methacrylic acid or their simple C₁₋₃₀ aromatic or aliphatic esters or hydroxy esters, which may be copolymerized with monomers such as vinyl pyrrolidone, acrylamide, vinyl acetate, acetate, vinyl isodecanoate, styrene, and olefins such as ethylene, propylene, butene, pentene, decene, hexadecene, and so on. The synthetic polymers may be homo- or copolymers of the monomer units mentioned. In addition, one or more of the above mentioned monomers may be copolymerized with various organic compounds such as polyalkylene glycols, paraffinic hydrocarbons, alkoxylated alcohols and the like. Examples of such polymers include acrylates copolymer, acrylamides copolymer, acrylates/octylacrylamide copolymer, acrylates/tris (trimethylsiloxy) silylpropyl methacrylate copolymer, methacrylate copolymer,

3. Vinyl Polymers

Also suitable are various types of vinyl homo- or copolymers from vinyl monomers such as vinyl acetate, vinyl propionate, vinyl pyrrolidone, which may be copolymerized with other monomers such as itaconic acid, acrylates or methacrylates as mentioned above, ethylene, propylene, butylene, and so on. In one preferred embodiment of the invention the film former is PVP which is soluble in the aqueous phase of the emulsion.

4. Silicone Resins

Silicone resins are also suitable film formers. They are generally highly crosslinked structures comprising combinations of M, D, T, and Q units. The term “M” means a monofunctional siloxy unit having the general formula:

[Si—(CH₃)₃—O]_{0.5}

In cases where the M unit is other than methyl (such as ethyl, propyl, ethoxy, etc.) the M unit may have a prime after it, e.g. M′.

The term “D” means a difunctional siloxy unit having the general formula:

[Si—(CH₃)₂—O]_{1.0}

The difunctional unit may be substituted with alkyl groups other than methyl, such as ethyl, propyl, alkylene glycol, and the like, in which case the D unit may be referred to as D′, with the prime indicating a substitution.

The term “T” means a trifunctional siloxy unit having the general formula:

[Si—(CH₃)—O]_{1.5}

The trifunctional unit may be substituted with substituents other than methyl, in which case it may be referred to as T′.

The term “Q” refers to a quadrifunctional siloxy unit having the general formula:

[Si—O—]_{2.0}

The silicone resins that may be used as film formers in the compositions of the invention preferably comprise highly crosslinked combinations of M, T, and Q units. Examples of such resins include trimethylsiloxysilicate which can be purchased from Dow Corning Corporation as 749 Fluid, or from GE Silicones under the SR-1000 trademark. Also suitable is a silicone resin that contains a large percentage of T groups, such as MK resin sold by Wacker-Chemie, having the CTFA name polymethylsilsesquioxane.

5. Copolymers of Silicone and Organic Monomers

Also suitable for use as the film formers are copolymers of silicone and organic monomers such as acrylates, methacrylates, and the like. Examples of such suitable film forming polymers include those commonly referred to as silicone acrylate or vinyl silicone copolymers, such as those sold by 3M under the brand name “Silicone Plus” polymers such as SA-70, having the CTFA name Polysilicone-7 and is a copolymer of isobutylmethacrylate and n-butyl endblocked polydimethylsiloxane propyl methacrylate; or VS-70 having the CTFA name Polysilicone-6, which is a copolymer of dimethylsiloxane and methyl-3 mercaptopropyl siloxane reacted with isobutyl methacrylate; or VS-80, having the CTFA name Polysilicone-8, which has the general structure:

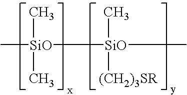

Where R represents the acrylates copolymer radical.

6. Natural Polymers

Also suitable are various types of natural polymers such as shellac, natural resins, chitin, and the like.

### V. Other Ingredients

The composition of the invention may comprise a variety of other ingredients including but not limited to those set forth herein. The composition may be in the form of lipstick, mascara, foundation makeup, blush, eyeliner, eyeshadow, concealer, skin cream or lotion, and the like.

A. Oils

If in the emulsion form, the composition preferably comprises one or more oils. If present, suggested ranges are from about 0.01 to 85%, preferably from about 0.1 to 80%, more preferably from about 0.5 to 75%. The term “oil” means a pourable liquid at room temperature. The oils may be volatile or non-volatile.

1. Volatile Oils

Suitable volatile oils generally have a viscosity ranging from about 0.5 to 5 centistokes 25° C. and include linear silicones, cyclic silicones, paraffinic hydrocarbons, or mixtures thereof.

(a). Volatile Silicones

Cyclic silicones are one type of volatile silicone that may be used in the composition. Such silicones have the general formula:

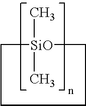

where n=3-6, preferably 4, 5, or 6.

Also suitable are linear volatile silicones, for example, those having the general formula:

(CH₃)₃Si—O—[Si(CH₃)₂—O]ₙ—Si(CH₃)₃

where n=0, 1, 2, 3, 4, or 5, preferably 0, 1, 2, 3, or 4.

Cyclic and linear volatile silicones are available from various commercial sources including Dow Corning Corporation and General Electric. The Dow Corning linear volatile silicones are sold under the tradenames Dow Corning 244, 245, 344, and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Suitable branched volatile silicones include alkyl trimethicones such as methyl trimethicone, a branched volatile silicone having the general formula:

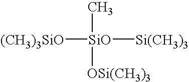

Methyl trimethicone may be purchased from Shin-Etsu Silicones under the tradename TMF-1.5, having a viscosity of 1.5 centistokes at 25° C.

(b). Volatile Paraffinic Hydrocarbons

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60 to 260° C., and a viscosity of less than about 10 cst. at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable.

2. Non-Volatile Oils

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to:

(a). Esters

Suitable esters are mono-, di-, and triesters. The composition may comprise one or more esters selected from the group, or mixtures thereof.

(i). Monoesters

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.

(ii). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

(iii). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described in the C.T.F.A. Cosmetic Ingredient Dictionary and Handbook, Eleventh Edition, 2006, under the classification of “Esters”, the text of which is hereby incorporated by reference in its entirety.

(b). Hydrocarbon Oils

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.

(c). Glyceryl Esters of Fatty Acids

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(d). Nonvolatile Silicones

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

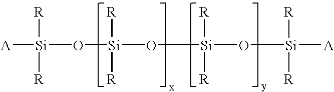

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814.

B. Surfactants

Surfactants may be present in the compositions preferably those that are nonionic and aid in formation of stabilized emulsions. Suitable surfactants may be silicone based or organic nonionic surfactants.

1. Silicone Surfactants

One type of silicone surfactant that may be used in the composition of the invention is generally referred to as dimethicone copolyol or alkyl dimethicone copolyol. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

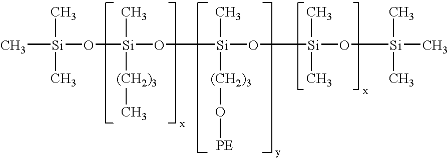

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

2. Organic Nonionic Surfactants

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000.

Also suitable as nonionic surfactants are formed by the reaction of a carboxylic acid with an alkylene oxide or with a polymeric ether. The resulting products have the general formula: where RCO is the carboxylic ester radical, X is hydrogen or lower alkyl, and n is the number of polymerized alkoxy groups. In the case of the diesters, the two RCO-groups do not need to be identical. Preferably, R is a C₆₋₃₀ straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

C. Particulates

The compositions of the invention may contain particulate materials in the form of pigments, inert particulates, or mixtures thereof. If present, suggested ranges are from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

1. Powders

The particulate matter may be colored or non-colored (for example white) non-pigmented powders. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

2. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

D. Aqueous Phase Gelling Agents

The composition may comprise one or more aqueous phase gelling agents. If present, suggested ranges are from about 0.01 to 40%, preferably from about 0.05 to 35%, more preferably from about 0.1 to 30% by weight of the total composition. Examples of such agents include polysaccharides such as A variety of polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

The compositions may additionally contain preservatives, humectants, peptides, fibers, and other non-essential ingredients.

The invention will be described in connection with the following examples which are set forth for the purposes of illustration only.

### EXAMPLE 1

Mascara compositions, in oil-in-water emulsion form, were prepared as follows:

| | % by weight | | phase |
|---|---|---|---|
| | 1 | 2 | A |
| D.I. Water | 20 | 20 | A |
| Silk amino acids | — | 0.01 | E |
| Acacia Senegal gum | 0.25 | 0.25 | A |
| Stearamide MEA-Stearate | 3.75 | 3.75 | C |
| Aminomethyl propanediol | 0.5 | 0.5 | B |
| Magnesium aluminum silicate | 0.15 | 0.15 | B |
| Glyceryl stearate/PEG-100 stearate | 0.63 | 0.63 | C |
| Polyurethane-1 | 8.6 | 8.6 | D |
| Sorbitan tristearate | 1.25 | 1.25 | C |
| Hydrolyzed jojoba protein | — | 0.01 | D |
| Hydroxystearic acid/polyglyceryl-10 | 1.64 | — | C |
| dodecabehenate | | | |
| Phenoxyethanol | 0.2 | 0.6 | E |
| Disodium EDTA | 0.1 | — | E |
| Isododecane | 16 | 10.5 | C |
| Microcrystalline wax | — | 1 | C |
| PTFE | — | 0.01 | B |
| Caprylyl glycol/phenoxyethanol/hexylene | — | 1 | E |
| glycol | | | |
| Phenoxyethanol/caprylyl glycol/potassium | 0.74 | — | E |
| sorbate/water/hexylene glycol | | | |
| Water/polyaminopropyl biguanide | 0.075 | — | E |
| Glyceryl hydrogenated rosinate | 5 | 5 | C |
| Ammonium hydroxide | 0.25 | 0.25 | B |
| Methyl trimethicone | — | 6.25 | C |
| Water/PVP/black iron oxides/silica/kaolin | 20 | 20 | B |
| Hydroxyethylcellulose | 0.1 | 0.1 | B |
| Coffee Seed extract | — | 0.01 | E |
| Stearic acid | 4.5 | 4.5 | C |
| Silica | 0.5 | 0.5 | B |
| Polypropylene | — | 9.05 | C |
| Pantothenic acid polypeptide | — | 0.01 | E |
| Potassium sorbate | 0.09 | — | E |
| Kaolin | 2 | 2 | B |
| Glyceryl hydrogenated rosinate/glyceryl | 5.05 | — | C |
| diisostearate/hydrogenated | | | |
| rosinate/phytostearyl-isostearyl-cetyl- | | | |
| stearyl-behenyl dimer dilinoleate | | | |
| Tocopheryl acetate | — | 0.01 | C |
| Water | QS 100 | QS100 | D |

The compositions were prepared by sprinkling the acacia gum and magnesium aluminum silicate into water using a high shear mixer and mixing for 30 minutes while increasing the temperature to 85° C. The Phase B ingredients were added with agitation taking care to ensure that all materials were adequately wetted. The batch was mixed for 15 minutes until dispersed. The Phase C ingredients were heated to 85° C. until completely melted, then slowly added to the batch with high speed agitation. The batch was mixed for 20 minutes, then cooled slowly to 70° C. Phase C was added to the batch with high speed agitation and mixed for 20 minutes then cooled slowly to 70° C. The batch was them mixed using a sweep mixer while cooling to 45° C. The phase D ingredients were combined with the batch and mixed for 10 minutes. The batch was cooled to 40° C. and the remaining phase E ingredients added. The batch was cooled while mixing to 28° C., then poured into containers.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. A aqueous based composition comprising at least one clear or translucent non-amidated structuring agent, at least one film forming polymer solubilized or dispersed in the aqueous phase, wherein the composition is substantially free of waxes.

2. The composition of claim 1 wherein the non-amidated structuring agent is selected from polyolefins, block copolymers, hydroxystearic acids, polysaccharides, rosin or rosin derivatives, or mixtures thereof.

3. The composition of claim 1 wherein the non-amidated structuring agent comprises polypropylene prepared by metallocene catalysis.

4. The composition of claim 1 wherein the non-amidated structuring agent comprises 12-hydroxystearic acid, hydrogenated glyceryl rosinate, glyceryl rosinate, or mixtures thereof.

5. The composition of claim 1 which is in the form of an oil in water emulsion.

6. The composition of claim 1 which is in the form of a water in oil emulsion.

7. The composition of claim 1 wherein the waxes are vegetable waxes.

8. The composition of claim 1 which is a mascara.

9. The composition of claim 1 wherein the film forming polymer comprises polyurethane.

10. The composition of claim 1 wherein the film forming polymer comprises PVP.

11. The composition of claim 1 wherein the non-amidated structuring agent is selected from 12-hydroxystearic acid, hydrogenated glyceryl rosinate, glyceryl rosinate, or mixtures thereof, the film former polymer is selected from PVP, polyurethane, or mixtures thereof, and the waxes are vegetable waxes.

12. The composition of claim 1 which is a mascara composition comprising from about 0.01 to 99%, preferably from about 0.05 to 95%, preferably from about 0.5 to 95% by weight of the total composition of water and from about 0.01 to 99%, preferably from about 0.05 to 95%, preferably from about 0.5 to 95% by weight of the total composition of oil.

13. The composition of claim 12 further comprising a linear, branched, or cyclic volatile silicone.

14. The composition of claim 13 further comprising a cellulose polymer.

15. The composition of claim 12 further comprising a volatile paraffinic hydrocarbon.

16. A method for making up keratinous surfaces comprising applying to the surface an aqueous based composition comprising at least one clear or translucent non-amidated structuring agent, at least one film forming polymer solubilized or dispersed in the aqueous phase, wherein the composition is substantially free of waxes.

17. The method of claim 16 wherein the keratinous surface is eyelashes.

18. The method of claim 17f wherein the composition additionally comprises pigments and powders.

19. The method of claim 16 wherein the waxes are natural waxes.

20. The method of claim 19 wherein the waxes are opaque.
